(19) United States
(12) Patent Application Publication
(10) Pub. No.: US 2008/0295581 A1
(43) Pub. Date: Dec. 4, 2008

(54) Method for the determination of aqueous polymer concentration in water systems

(75) Inventors: Li Zhang, Shanghai (CN); Caibin Xiao, Harleysville, PA (US); Yinhua Long, Shanghai (CN); Weiyi Cui, Piscataway, NJ (US); Bingzhi Chen, Shanghai (CN); Zhixin Zheng, Fujian (CN)

Correspondence Address: WEGMAN, HESSLER & VANDERBURG, 6055 ROCKSIDE WOODS BOULEVARD, SUITE 200, CLEVELAND, OH, 44131, US

(73) Assignee: General Electric Company, Schenectady, NY (US)

(21) Appl. No.: 11/809,345

(22) Filed: May 31, 2007

(51) Int. Cl. G01N 33/00; C08J 5/22

(52) U.S. Cl. 73/61.43; 521/27

(57) ABSTRACT

The concentration of an anionically charged polymer in an aqueous solution is determined with a thin solid film having a polymer matrix and a cationic dye. A sample of an aqueous solution containing at least one anionically charged polymer to be tested is applied to the film sensor. The absorbance of the film sensor is measured after the sample has been applied. The absorbance of the film sensor is then compared with a calibration curve of the absorbance of samples containing known concentrations of the anionically charged polymers to determine the concentration of anionically charged polymer in the sample.

### BACKGROUND OF THE INVENTION

1. Field of The Invention

The invention relates generally to the detection of water-soluble polymers in industrial water systems such as cooling and boiler water systems, and more specifically to a method of determining the concentration or availability of anionic water-soluble polymers in industrial water systems using a solid film sensor.

2. Description of Related Art

Water is used in a number of industrial water systems such as cooling and boiler water systems. Municipal or untreated water contains impurities which can affect heat transfer, fluid flow or cause corrosion of system equipment. For example, metal cations such as calcium, magnesium, barium and sodium are often present in untreated water. When the water contains an excess of these impurities, precipitates can form on equipment surfaces in the form of scales or deposits. The presence of these scales or deposits adversely affects the rate of heat transfer, and therefore the efficiency of the system. Furthermore, the cleaning or removal of such scales or deposits is expensive and burdensome because it typically requires a shutdown of the system. Accordingly, before the water is utilized for cooling or steam purposes, it is desirably treated with appropriate chemicals in order to inhibit scale formation.

A number of chemicals have been provided to reduce or inhibit scale and deposit formation in industrial water systems. For example, it is known to add anionic water-soluble polymers to the water. One particularly useful water-soluble polymer is HPS-I; although other water-soluble polymers such as AEC and APES are in use as well. However, the employment of water-soluble polymers in industrial water systems presents its own set of problems because the concentration of the polymers in the water must be carefully monitored. For example, if too little of the polymer is employed, scaling and deposition will occur. In contrast, if too high a concentration of the polymer is employed, then the cost/performance efficiency of the system is adversely affected. As with other methods of chemically treating aqueous systems, there is an optimal concentration of treatment chemicals that should be maintained.

Several methods for determining the concentration of water-soluble polymers in aqueous systems are available. For example, there are several colorimetric methods for determination of polyelectrolytes using dyes. One example is U.S. Pat. No. 6,214,627 issued to Ciota et al. In addition, there is a Hach polyacrylic acid method that uses iron thiocyanate chelation to detect calibration based on polyacrylic acid. Generally, these methods require a complicated, multi-step operation procedure and are difficult to carry out in the field. Other methods, such as the one disclosed in U.S. Pat. No. 5,958,778 issued to Johnson et al., use luminol-tagged polymers in combination with fluorescent or chemiluminescent detection techniques to monitor the industrial waters. Also, there is a turbidity method that relies on the formation of insoluble compounds for determining the concentration of water soluble polymers. This method is lengthy and is susceptible to inaccuracies.

Thus, there exists a strong need for simplified sensors and test methods that can easily be used to determine the concentration of water soluble polymers in aqueous systems with high reproducibility, decreased response to interferences, and enhanced stability.

### SUMMARY OF THE INVENTION

In one aspect, the invention is directed to a method for measuring the concentration of an anionically charged polymer in an aqueous solution. The method includes the steps providing a thin solid film sensor comprising a polymer matrix and a cationic dye. A sample of an aqueous solution containing at least one anionically charged polymer to be tested is applied to the film sensor. After the sample has been applied, the absorbance of the film sensor is measured. The absorbance of the film sensor is then compared with a calibration curve of the absorbance of samples containing known concentrations of the anionically charged polymers to determine the concentration of anionically charged polymer in the sample.

Another aspect of the invention is directed to a solid film sensor for measuring the concentration of an anionically charged polymer in an aqueous solution comprising a polymer matrix and a cationic dye. The cationic dye is selected from the group consisting of Dimethyl Methylene Blue, Basic Blue 17, and New Methylene Blue N.

The present invention and its advantages over the prior art will become apparent upon reading the following detailed description and the appended claims with reference to the accompanying drawings.

### BRIEF DESCRIPTION OF THE DRAWINGS

The above mentioned and other features of this invention will become more apparent and the invention itself will be better understood by reference to the following description of embodiments of the invention taken in conjunction with the accompanying drawings, wherein:

FIG. 1 depicts spectrums of water samples with different amounts of an anionic polymer after reaction on a solid film sensor;

FIG. 2 depicts plots of absorbance vs. concentration for the anionic polymer plotting absorbance vs. HPS-I concentration at 650 nm;

FIG. 3 depicts a calibration curve for HPS-I plotting the delta absorbance of 575 nm minus 525 nm vs. HPS-I concentration;

FIG. 4 depicts a calibration curve for HPS-I plotting the delta absorbance of red minus green vs. HPS-I concentration; and

FIG. 5 depicts a calibration curve for HPS-I at 575 nm plotting absorbance vs. HPS-I concentration.

Corresponding reference characters indicate corresponding parts throughout the views of the drawings.

### DETAILED DESCRIPTION OF THE INVENTION

The invention will now be described in the following detailed description with reference to the drawings, wherein preferred embodiments are described in detail to enable practice of the invention. Although the invention is described with reference to these specific preferred embodiments, it will be understood that the invention is not limited to these preferred embodiments. But to the contrary, the invention includes numerous alternatives, modifications and equivalents as will become apparent from consideration of the following detailed description.

Disclosed are improved solid film sensor compositions and methods of detecting anionic water-soluble polymers in industrial water systems. The method disclosed herein is particularly well suited for quickly and accurately determining the concentrations of anionic polymer corrosion or scale inhibitors in aqueous systems, including but not limited to boilers, cooling towers, evaporators, gas scrubbers, kilns and desalination units. Polymers capable of being detected by the method of the invention include, but are not limited to, polyacrylic acid moiety polymers, polysufonated polymers and maleic anhydride polymers. Specific examples of some contemplated anionic polymers are HPS-I (from GE Betz of Trevose, Pa.), AEC, and APES.

Applicants have discovered that solid film sensors containing certain metachromatic dyes are suitable for use in calorimetrically determining the concentration of anionic polymers in aqueous systems. Certain dyes undergo a unique color change upon interaction with polyionic compounds in solution. When anionic polymers contact the metachromatic dye in the film sensor, the dye molecules align with the anionic charges on the polymers, resulting in a shift in the wavelength of maximum absorbance of the dye molecule. This shift is observable as a color change of the film sensor. The concentrations of anionic polymers in aqueous solutions can be determined calorimetrically by applying a sample of the aqueous solution to the film sensor and measuring the absorbance of the film sensor at a specified wavelength. The measured absorbance is then compared to the absorbance of standards having known concentrations of the species being measured.

The ink composition needed to make the film sensor comprises a polymer-based composition generally including a metachromatic dye, a polymer matrix or combination of polymer matrices, and auxiliary minor additives, wherein the film is produced from a solution of the components in a common solvent or solvent mixture. Examples of additives are surfactants and antifoaming agents.

The metachromatic dye is a cationic dye with a phenothiazine structure. It has been found that Dimethyl Methylene Blue, Basic Blue 17, and New Methylene Blue N are especially suitable metachromatic dyes. Table 1 illustrates the structures of these dyes.

TABLE 1
List of Dyes and Their Structures

| | |
|---|---|
| DimethylMethyleneBlue | 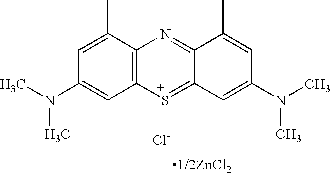 |
| Basic Blue17 | 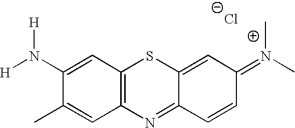 |
| New Methy-lene Blue N | 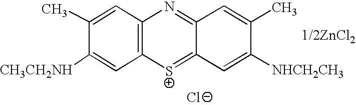 |

The matrix of the ink compositions can be divided into two types according to the solubility of the film sensors in water samples. A first matrix is insoluble in water and the other is a completely soluble matrix. The dye is added into either of the two types of matrices to form the ink composition.

For the water soluble matrix, various water-soluble polymers may be employed. The water-soluble resin includes, for example, polyvinyl alcohol resins in which the hydroxyl groups are hydrophilic structural units [e.g., polyvinyl alcohol (PVA), acetoacetyl-modified polyvinyl alcohol, cation-modified polyvinyl alcohol, anion-modified polyvinyl alcohol, silanol-modified polyvinyl alcohol, polyvinylacetal], cellulose resins [methyl cellulose (MC), ethyl cellulose (EC), hydroxyethyl cellulose (HEC), carboxymethyl cellulose (CMC), hydroxypropyl cellulose (HPC), hydroxyethylmethyl cellulose, hydroxypropylmethyl cellulose], chitins, chitosans, starches, ether bond-having resins [polyethylene oxide (PEO), polypropylene oxide (PPO), polyethylene glycol (PEG), polyvinyl ether (PVE)], and carbamoyl group-having resins [polyacrylamide (PAA), polyvinylpyrrolidone (PVP), polyacrylic hydrazide]. The water-soluble polymer is solved in water and prepared to stock solution with appropriate viscosity for preparing film.

The matrix may include about 0.01 to about 10% of a surfactant. In a preferred embodiment, the surfactant is TWEEN-20 or TRITON X-100. For example, 0.05% of TWEEN-20 may desirably be employed in the invention. In another embodiment, the releasing component is substantially free of a surfactant.

The water-soluble matrix further can include an antifoaming agent with a concentration ranging from 0.1 to 10% by weight, with typical amounts being less than 5 percent by weight, and desirably less than 0.5 percent by weight. Desirably, the antifoaming agent is an organic silicone antifoam. In preferred embodiments, the antifoam agent is Sag 638 SFG or Y-17236 from Momentive Performance Materials of Wilton, Conn.

In one suitable ink matrix, between about 7 g-10 g of the polymer stock solution is used. Between 0.2-0.8 g Tween-20 and 0-1 g Sag 638 SFG are mixed and stirred at room temperature for at least two hours. The dye is added to form a ratio of dye to matrix of ink from 0.01:10 to 0.06:10.

The insoluble matrix uses a polymer desirably selected from the cellulose ester plastics, including for example, cellulose acetate, cellulose acetate butyrate and cellulose porpionate. In one preferred embodiment, cellulose acetate (Mw over 10,000) is used. The polymer is dissolved in a solvent or a combination of organic solvents. Representative examples of some suitable solvents include cyclohexanone, acetone, xylene, toluene, i-propanol, di(ethlyene glycol)methyl ether, poly(ethylene glycol)dimethyl ether, N,N-dimethylformamide (DMF), tethrahydrofurane (THF), methyl ethyl ketone, propylene glycol monomethyl ether, methyl butyl ketone, ethyl acetate, n-butyl acetate, dioxane, propyl cellosolve, butyl cellosolve, and other cellosolves. Some solvent mixtures can be used as well.

In one suitable ink matrix, cellulose acetate in solvents (7%-15% cellulose acetate) is mixed and stirred at room temperature for over 24 hours. The dye is added such that the ration of dye to matrix of ink is from 0.01:10 to 0.06:10.

A sensor film is formed from the ink using known deposition methods. Non-limiting examples of these deposition methods include ink-jet printing, spray coating, screen-printing, array microspotting, dip coating, solvent casting, draw coating and any other known in the art. In one embodiment, a polymer film is made with a final film thickness desirably between about 0.1 and about 200 microns, more preferably 0.5-100 microns and more preferably 1-50 microns.

In order to determine the concentration or amount of available anionic polymer in an industrial water system, it is first necessary to generate a calibration curve for each polymer of interest. Calibration curves are generated by preparing various samples of water containing known amounts of polymer, applying the samples to film sensors, and measuring the absorbance of the samples. For purposes of this work, absorbance is being reported as absorbance difference. Absorbance difference is the difference between the absorbance of the film sensor by itself and the absorbance of the film sensor after a sample of water being tested is applied to the film sensor. The calibration curve is then a plot of this absorbance difference vs. the known concentration of polymer in the sample. Once created, the calibration curve can be used to determine how much polymer is present in a sample by comparing the measured absorbance difference of the sample with the calibration curve and reading the amount of polymer present off of the curve. In order to use the calibration curve, the device used to measure absorbance must be the same or operate on similar conditions as the device that was used to create the calibration curve.

The absorbencies may be measured using any suitable device known in the art to measure absorbance. Such suitable devices include, but are not limited to, colorimeters, spectrophotometers, color-wheels, and other types of known color-comparitor measuring tools. In one embodiment, measurements of optical response can be performed using an optical system that included a white light source (such as a Tungsten lamp available from Ocean Optics, Inc. of Dunedin, Fla.) and a portable spectrometer (such as Model ST2000 available from Ocean Optics, Inc. of Dunedin, Fla). Other suitable spectrophotometers include the DR/2010 spectrophotometer, which is available from Hach Company of Loveland, Colo. and the DR/890 Colorimeter, which is also available from Hach Company. Other known methods of measuring the response may also be used.

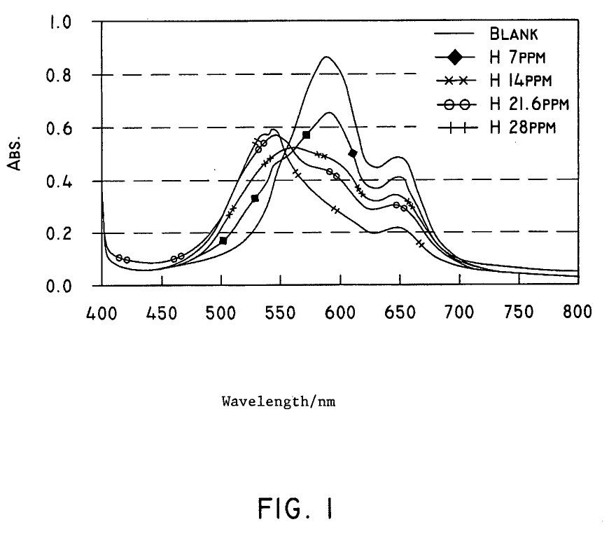

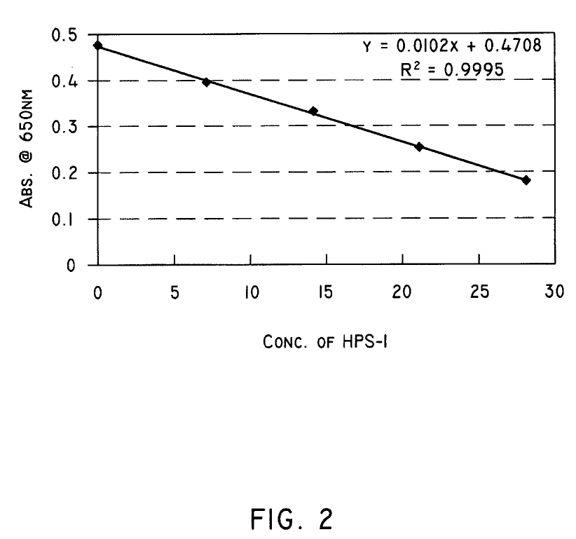

FIG. 1 shows the spectrums of a water sample with different amounts of an anionic polymer (e.g., H stands for HPS-I polymer from GE Betz of Trevose, Pa.) after reaction on solid film sensors. FIG. 2 illustrates the calibration curve for the absorbance at 650 nm. Once created, calibration curves can be repetitively used for determining the concentration of the desired anionic polymer in the sample of water being tested. Calibration curves are easily generated, as described above, and can be posted on site or stored electronically for determining the concentration of the desired anionic polymer in the sample of water being tested.

In one embodiment, in order to determine the concentration of anionic polymer in a sample of water using this method, between about 30 μL and about 50 μL of sample, desirably about 35 μl of the water sample is added onto the film sensor. However other amounts are contemplated without departing from the scope of the invention. The anionic polymer in the sample is then allowed to react with the film sensor for a period of time of desirably between about 0.5 and 7 minutes, preferably between about 1 and about 5 minutes. It has been found that the reaction is usually complete in about 3 minutes, making any absorbance measurement taken at about 3 minutes and thereafter accurate. It has been found that this accurate absorbance measurement remains essentially stable for the first seven minutes of time, with minor fluctuations occurring after the first seven minutes.

Once the absorbance of the film sensor is measured (usually as the absorbance difference described above), it is compared with calibration curves that show the standard absorbance of solutions containing known amounts of the specific anionic polymer. In this way, the amount of anionic polymer present in the sample can be determined. In one yet another embodiment, the measurement is done continuously before water exposure, during water exposure, and after water exposure.

The present disclosure will now be described more specifically with reference to the following examples. It is to be noted that the following examples are presented herein for purpose of illustration and description; they are not intended to be exhaustive or to limit the disclosure to the precise form disclosed.

### EXAMPLE 1

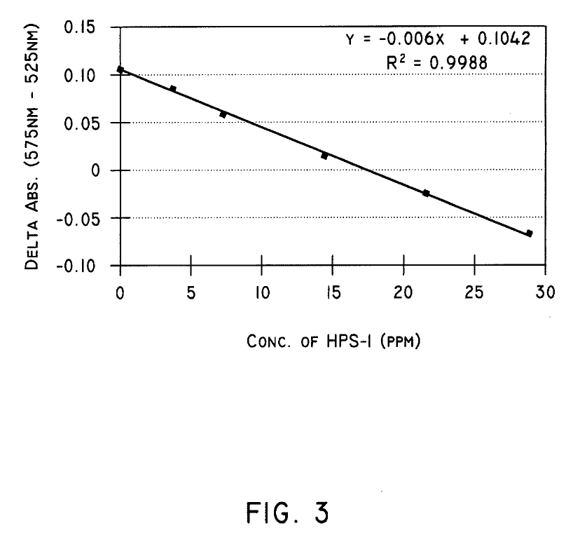

A polymer matrix comprising 10 g of PEO (Mw=200,000) in water (14.3%), 2.4 g of PEG (Mw=2,000) in water (60%), 0.25 g Tween 20, 0.125 g antifoam Sag 638 SFG and 50 mg DMMB were mixed and stirred at room temperature until the entire solid was dissolved. The film was prepared by screen-printing and dried at 70° C. for 10 minutes. The film was tested using a HPS-I standard solution. The spectra were read using a microplate reader at 575 nm and 525 nm and the delta absorbance of 575 nm minus the 525 nm was plotted as a function of HPS-I concentration. FIG. 3 illustrates the calibration curve obtained.

### EXAMPLE 2

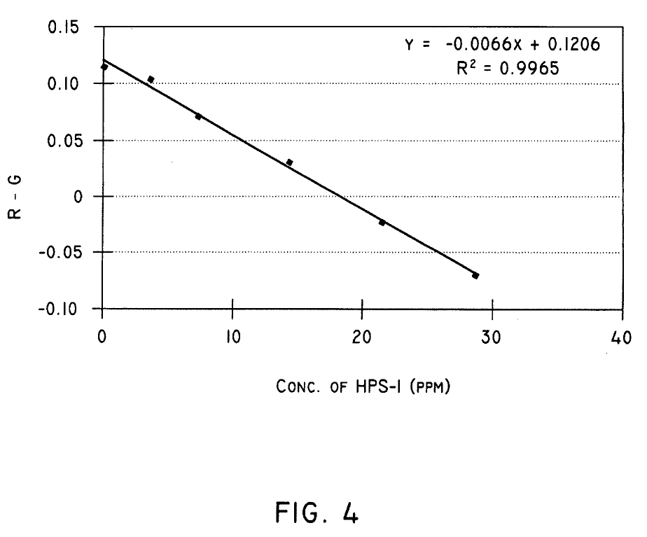

10 g of 33.3% PAA (Mw=5,000) in mixture of H₂O and ethylene glycol (1:1), 0.086 g Tween 20 and 20 mg DMMB were mixed and stirred at room temperature until the entire solid was dissolved. The film was prepared by screen-printing and dried at 70° C. for 10 minutes. The film was tested using a HPS-I standard solution. The spectra were read by tricolor (Red, Green, Blue) LED and the delta absorbance of Red minus Green was plotted as a function of HPS-I concentration. FIG. 4 illustrates the calibration curve obtained.

### EXAMPLE 3

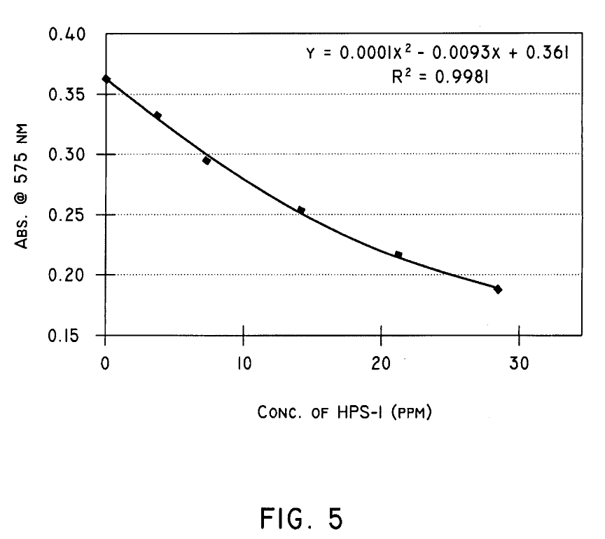

2.4 g (13.4%) Cellulose acetate in di(ethlyene glycol)methyl ether, 7.6 g cellulose acetate in poly(ethylene glycol)dimethyl ether, 15 mg CTAB, and 120 mg DMMB were mixed and stirred at room temperature until the entire solid was dissolved. The film was prepared by screen-printing and dried at 70° C. for 10 minutes. The film was tested using a HPS-I standard solution. The spectra were read with a microplate reader at 575 nm and plotted as a function of HPS-I concentration. FIG. 5 illustrates the calibration curve obtained.

While the disclosure has been illustrated and described in typical embodiments, it is not intended to be limited to the details shown, since various modifications and substitutions can be made without departing in any way from the spirit of the present disclosure. As such, further modifications and equivalents of the disclosure herein disclosed may occur to persons skilled in the art using no more than routine experimentation, and all such modifications and equivalents are believed to be within the scope of the disclosure as defined by the following claims.

What is claimed is:

1. A method for measuring the concentration of an anionically charged polymer in an aqueous solution that comprises the steps of:
providing a thin solid film sensor comprising a polymer matrix and a cationic dye;
applying a sample of an aqueous solution containing at least one anionically charged polymer to be tested to the film sensor;
measuring the absorbance of the film sensor after the sample has been applied; and
comparing the absorbance of the film sensor with a calibration curve of the absorbance of samples containing known concentrations of the anionically charged polymers to determine the concentration of anionically charged polymer in the sample.

2. The method of claim 1 wherein the cationic dye is selected from the group consisting of Dimethyl Methylene Blue, Basic Blue 17, and New Methylene Blue N.

3. The method of claim 2 wherein the film sensor has a thickness of less than 50 microns.

4. The method of claim 2 wherein the film sensor is made from a polymer stock solution containing a surfactant.

5. The method of claim 4 wherein the surfactant is a cationic surfactant.

6. The method of claim 4 wherein the surfactant is a nonionic surfactant.

7. The method of claim 6 wherein the nonionic surfactant is Tween 20 with concentration ranged from 0.01 to 10% by weight of the total polymer stock solution.

8. The method of claim 2 wherein the film sensor is made from a polymer stock solution containing antifoaming agent.

9. The method of claim 8 wherein the antifoaming agent is organic silicone antifoaming agent with concentration ranging from 0.1 to 10% by weight.

10. The method of claim 2 wherein the polymer matrix of the film sensor is made from a polymer stock solution between about 7 g-10 g with 0.2-0.8 g Tween-20 and 0-1 g Sag 638 SFG and the dye is added to form a ratio of dye to matrix from 0.01:10 to 0.06:10.

11. The method of claim 2 wherein the anionically charged polymer to be measured is selected from the group consisting of HPS-I, AEC, and APES.

12. A solid film sensor for measuring the concentration of an anionically charged polymer in an aqueous solution comprising a polymer matrix and a cationic dye, wherein the cationic dye is selected from the group consisting of Dimethyl Methylene Blue, Basic Blue 17, and New Methylene Blue N.

13. The film sensor claim 12 wherein the film sensor has a thickness of less than 50 microns.

14. The film sensor claim 12 wherein the film sensor comprises a surfactant and an antifoaming agent.

15. The film sensor claim 14 wherein the surfactant is cationic surfactant.

16. The film sensor claim 15 wherein the surfactant is nonionic surfactant.

17. The film sensor claim 16 wherein the nonionic surfactant is Tween 20 with concentration ranging from 0.01 to 10% weight percent of the total polymer stock solution.

18. The film sensor claim 14 wherein the antifoaming agent is an organic silicone antifoam agent.

19. The film sensor claim 18 wherein the polymer matrix of the film sensor is made from a polymer stock solution between about 7 g-10 g with 0.2-0.8 g Tween-20 and 0-1 g Sag.638 and the dye is added to form a ratio of dye to matrix from 0.01:10 to 0.06:10.

20. The film sensor of claim 12 wherein the anionically charged polymer to be measured is selected from the group consisting of HPS-I, AEC, and APES.
